(19) United States
(12) Patent Application Publication
(10) Pub. No.: US 2026/0283724 A1
(43) Pub. Date: Sep. 24, 2026

(54) PLATE AND SURGICAL ROBOT SYSTEM

(71) Applicant: Qoly Corporation, Tokyo (JP)

(72) Inventors: Hirofumi OWAKI, Tokyo (JP); Kazuhito WAKANA, Tokyo (JP); Natsuki MATSUMOTO, Tokyo (JP)

(73) Assignee: Qoly Corporation, Tokyo (JP)

(21) Appl. No.: 19/166,045

(22) PCT Filed: Mar. 22, 2024
(86) PCT No.: PCT/JP2024/011377

§ 371 Date: Sep. 17, 2025

(30) Foreign Application Priority Data
Mar. 31, 2023 (JP) 2023-059413

(51) Int. Cl. A61B 34/35; A61B 17/04; A61B 17/08; A61B 17/3211

(52) U.S. Cl. CPC A61B 34/35; A61B 17/0482; A61B 17/083; A61B 17/3211

(57) ABSTRACT

A plate includes a surgical tool placement portion that is a region on which a surgical tool is placed and sandwiched between an arc-shaped first outer edge and an arc-shaped second outer edge concentric with the first outer edge and having a small radius.

### FIELD

The present disclosure relates to a plate on which a surgical tool is placed and a surgical robot system.

### BACKGROUND

The surgery is performed using various surgical tools. In recent years, surgery supporting robots that perform surgery by being remotely operated have been studied. In the surgery supporting robot, an end effector attached to the distal end of the arm of the robot is substituted for the surgical tool. Therefore, the surgery is performed while replacing the end effector according to the situation of the surgery. It takes time to replace the end effector. Therefore, as the number of times of replacement of the end effector increases, the operation time increases, and the burden on the patient increases. Therefore, Patent Literature 1 discloses technology capable of reducing the number of times of replacement of the end effector by providing a plurality of end effectors in a tool provided at the distal end of the arm of a robot.

### CITATION LIST

### Patent Literature

Patent Literature 1: JP 2021-153858 A

### SUMMARY

### Technical Problem

When a plurality of end effectors is provided, a tool provided at the distal end of the arm becomes large. Surgery is often performed in a narrow range, and it may be difficult to perform surgery with a large tool.

Therefore, the present disclosure proposes a plate that can contribute to shortening of the operation time by reducing the number of times of replacement of the end effector while reducing the size of the tool.

### Solution to Problem

A plate includes a surgical tool placement portion that is a region on which a surgical tool is placed and sandwiched between an arc-shaped first outer edge and an arc-shaped second outer edge concentric with the first outer edge and having a small radius.

### BRIEF DESCRIPTION OF DRAWINGS

FIG. 1 is a view illustrating a schematic configuration of a surgery supporting robot according to a first embodiment of the present disclosure.

FIG. 2 is a perspective view of an operation device according to the first embodiment.

FIG. 3 is a partially enlarged perspective view in which a distal end portion of an arm in the first embodiment is enlarged.

FIG. 4 is a partially enlarged plan view in which an affected part on which a plate according to the first embodiment is disposed is enlarged.

FIG. 5 is a cross-sectional view obtained by cutting along a line V-V illustrated in FIG. 4.

FIG. 6 is a cross-sectional view obtained by cutting along a line VI-VI illustrated in FIG. 4.

FIG. 7 is a partially enlarged plan view in which an affected part on which the plate according to the first embodiment is disposed is enlarged, and is a view for explaining a change in an imaging range due to rotation of a camera.

### DESCRIPTION OF EMBODIMENTS

Hereinafter, an embodiment of the present disclosure will be described in detail with reference to the drawings. Note that, a plate and a surgical robot system according to the present disclosure are not limited by this embodiment. In addition, in the present specification and the drawings, components having substantially the same functional configuration are basically denoted by the same reference signs, and redundant description is omitted.

One or more embodiments described below can each be implemented independently. On the other hand, at least some of the plurality of embodiments described below may be appropriately combined with at least some of other embodiments. The plurality of embodiments may include novel features different from each other. Therefore, the plurality of embodiments can contribute to solving different objects or problems, and can exhibit different effects.

The present disclosure will be described according to the following order of items.
1. First Embodiment
1-1. Overview of Surgery Supporting Robot
1-1-1. Configuration of Operation Device
1-1-2. Configuration of Plate
1-1-3. Configuration of Operating Device
1-2. Effects of First Embodiment
2. Supplementary Note

### 1. First Embodiment

### 1-1. Overview of Surgery Supporting Robot

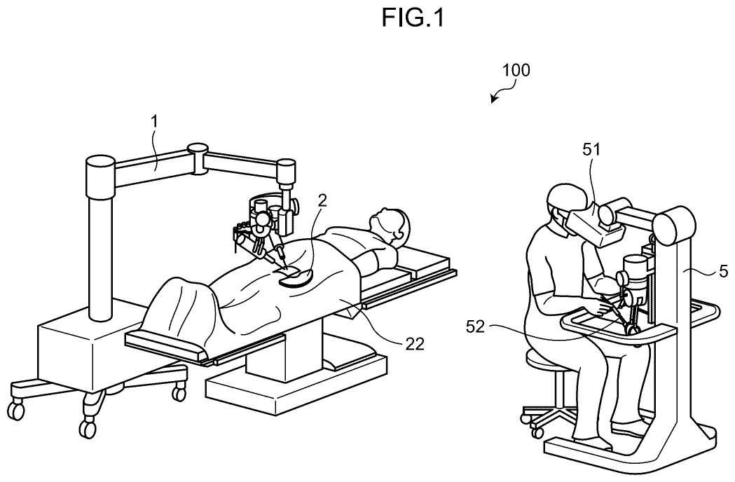

FIG. 1 is a view illustrating a schematic configuration of a surgery supporting robot according to a first embodiment of the present disclosure. A surgery supporting robot (surgical robot system) 100 includes an operation device 1, a plate 2, and an operating device 5. The operation device 1 is disposed in the vicinity of a patient undergoing surgery. The operation device 1 includes an arm to which a tool necessary for surgery is attached. The operating device 5 is disposed at a place away from the operation device 1. An arm and a tool provided in the operation device 1 can be operated by operating the operating device 5. A practitioner performing surgery operates the operating device 5 to cause the operation device 1 to perform surgery from a place away from the patient.

### 1-1-1. Configuration of Operation Device

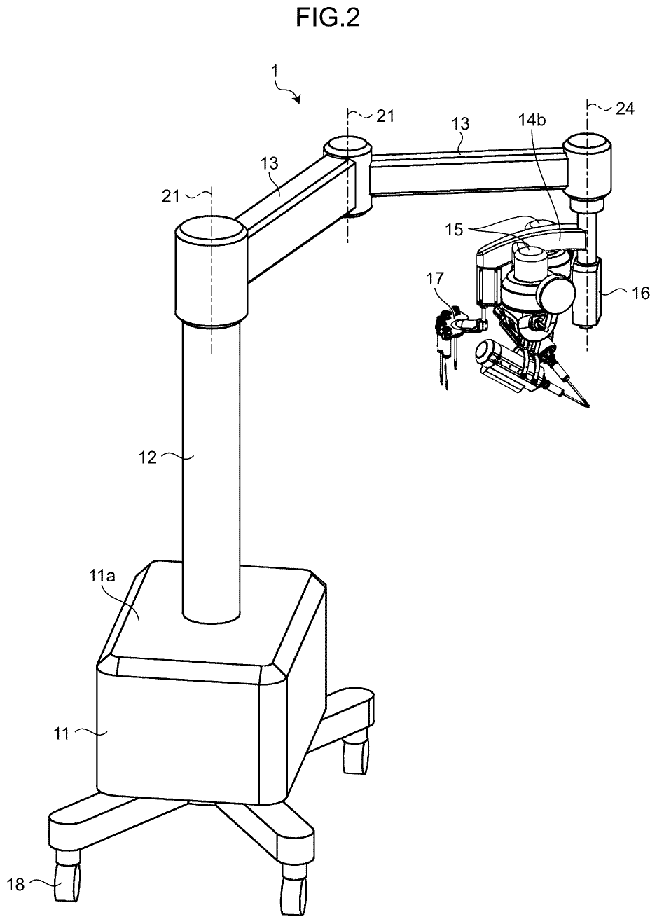

FIG. 2 is a perspective view of an operation device according to the first embodiment. The operation device 1 includes a base 11, a support column 12, an arm 13, a tool unit 15, a camera 16, and a tool providing unit 17.

The base 11 is a portion placed on a floor surface which is a placement surface. Wheels 18 are attached to the base 11. Attachment of the wheels 18 facilitates the movement of the operation device 1. The support column 12 is attached to a top surface 11a of the base 11. The support column 12 is a columnar member extending vertically. A plurality of arms 13 is provided, and the arms 13 are connected to each other so as to be rotatable about a rotation shaft 21.

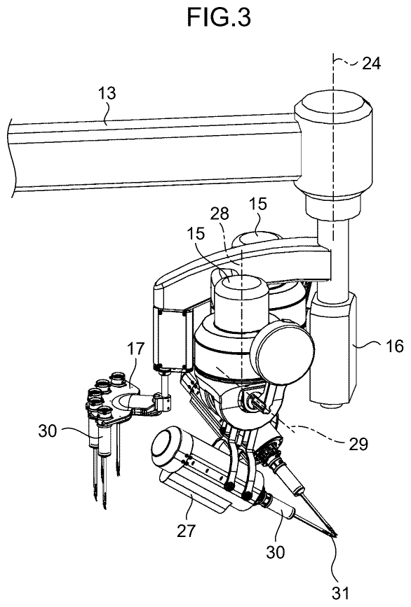

FIG. 3 is a partially enlarged perspective view in which a distal end portion of an arm in the first embodiment is enlarged. The tool unit 15 and the camera 16 are provided at the distal ends of the plurality of connected arms 13. The tool unit 15 and the camera 16 are rotatable about a drive shaft 24 provided at the distal end of the arm 13.

The tool unit 15 is provided with a base portion 25, a tool driving unit 27, and a tool 30. The base portion 25 rotates the tool driving unit 27 about a first drive shaft 28 extending vertically and a second drive shaft 29 extending horizontally. The tool 30 is detachably attached to the distal end of the tool driving unit 27. The tool driving unit 27 includes an actuator (not illustrated). When the actuator provided in the tool driving unit 27 is driven, an end effector 31 provided at the distal end of the tool 30 is driven.

The camera 16 captures an image of the lower side and transmits the captured image to the operating device 5. A range captured by the camera 16 is an operative field. During an operation, the tool driving unit 27 is driven so that the tool 30 falls within the operative field below the camera 16.

The tool providing unit 17 holds a plurality of tools 30 for replacement. In the surgery supporting robot 100, the end effector 31 provided at the distal end of the tool 30 serves as a substitute for the surgical tool. Therefore, in the surgery supporting robot 100, the surgery is advanced while replacing the tool 30 attached to the tool driving unit 27 and switching the end effector 31.

### 1-1-2. Configuration of Plate

As illustrated in FIG. 1, the plate 2 is disposed near an affected part where surgery is performed. A drape 22 is covered on a patient during surgery. An opening 22a is formed in the drape 22. A surgical site where the surgery is performed is exposed from the opening 22a.

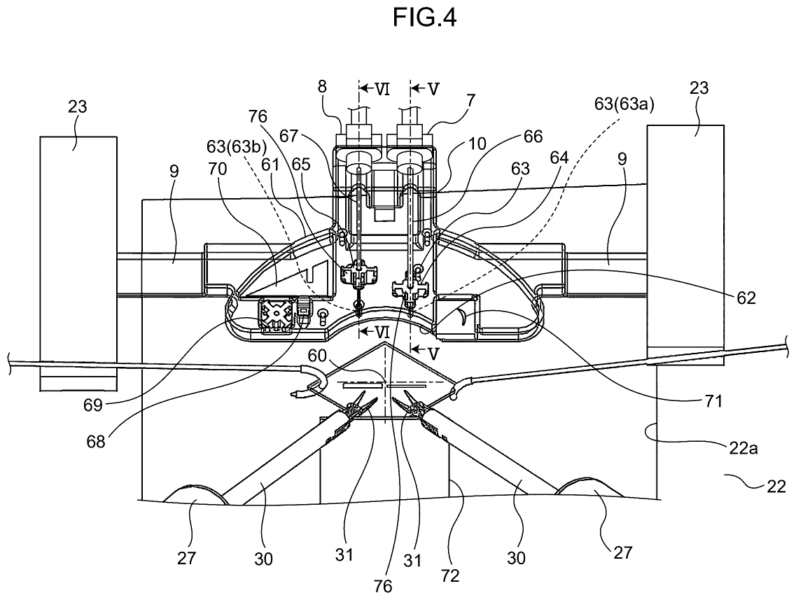

FIG. 4 is a partially enlarged plan view in which an affected part on which a plate according to the first embodiment is disposed is enlarged. The plate 2 is disposed near the surgical site exposed from the opening 22a of the drape 22. The plate 2 includes a surgical tool placement portion 6, a wiring fixing portion 7, a piping fixing portion 8, and an extension portion 9.

The surgical tool placement portion 6 is a region where a surgical tool is placed. The outer edge of the surgical tool placement portion 6 includes an arc-shaped first outer edge 61 and an arc-shaped second outer edge 62. The first outer edge 61 and the second outer edge 62 are formed in concentric arc shapes. The radius of the arc shape of the second outer edge 62 is smaller than the radius of the arc shape of the first outer edge 61. The surgical tool placement portion 6 is a region sandwiched between the first outer edge 61 and the second outer edge 62. The plate 2 is disposed such that a center 60 of the first outer edge 61 and the second outer edge 62 overlaps the center of the surgical site. As a result, the surgical tool placement portion 6 is disposed so as to surround the surgical site. The center of the surgical site is, for example, the center of an incised affected part as illustrated in FIG. 4.

A plurality of insertion portions 63 into which the distal end of the surgical tool is inserted is formed in the surgical tool placement portion 6. The insertion portion 63 is a hole having a bottom. The surgical tool to be inserted into the insertion portion 63 is, for example, a monopolar 64 which is an electronic scalpel. The monopolar 64 incises the affected part at the distal end. The monopolar 64 is used while being gripped by the end effector 31.

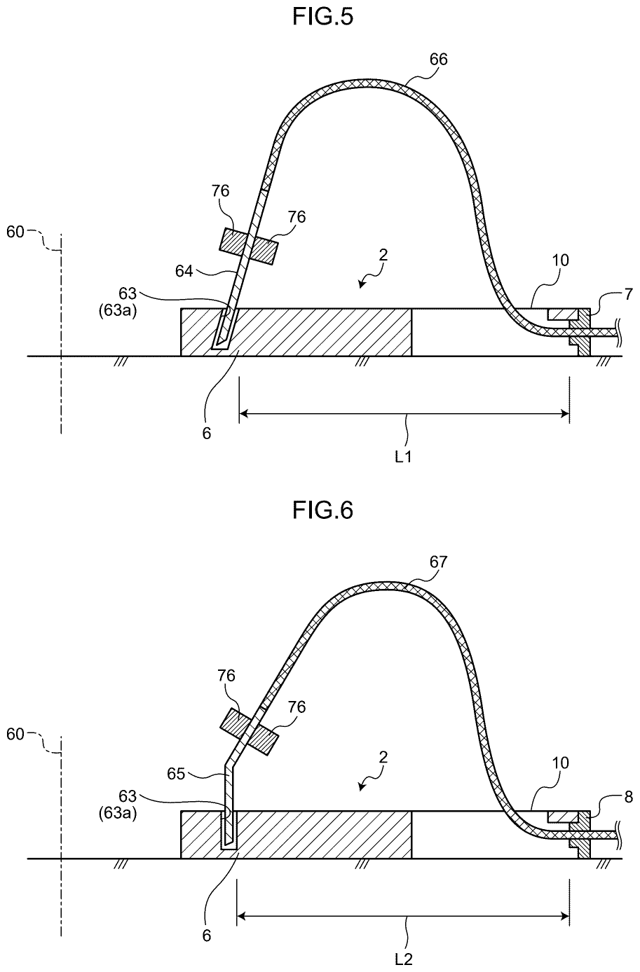

FIG. 5 is a cross-sectional view obtained by cutting along a line V-V illustrated in FIG. 4. As illustrated in FIG. 5, by being inserted into the insertion portion 63, the surgical tool is disposed in a state in which the longitudinal direction is closer to vertical than horizontal. Note that, in the following description, an arrangement in which the longitudinal direction is closer to vertical than horizontal is referred to as a vertical arrangement. In addition, an arrangement in which the longitudinal direction is closer to horizontal than vertical is referred to as a horizontal arrangement.

Among the plurality of insertion portions 63, a first insertion portion 63a into which the monopolar 64 is inserted is inclined so as to be away from the center 60 as it is away upward from the surface of the surgical tool placement portion 6. Therefore, the monopolar 64 inserted into the insertion portion 63 is also inclined so as to be away from the center 60 as it is away upward from the surface of the surgical tool placement portion 6.

Returning to FIG. 4, the surgical tool to be inserted into the insertion portion 63 is, for example, a discharge portion 65. Physiological saline is discharged from the distal end of the discharge portion 65. Cleaning or the like of the affected part is performed by the physiological saline discharged from the discharge portion 65. The discharge portion 65 is used while being gripped by the end effector 31.

FIG. 6 is a cross-sectional view obtained by cutting along a line VI-VI illustrated in FIG. 4. As illustrated in FIG. 6, among the plurality of insertion portions 63, a second insertion portion 63b into which the discharge portion 65 is inserted is a hole extending vertically. The discharge portion 65 inserted into the second insertion portion 63b is placed in a vertical arrangement. The discharge portion 65 is bent in the middle, and is inclined so as to be away from the center 60 as it is away upward from the surface of the surgical tool placement portion 6 at the root portion from the bent portion.

The monopolar 64 and the discharge portion 65 are provided with a gripped portion 76. The gripped portion 76 is a portion gripped by the end effector 32. The gripped portion 76 is formed so as to protrude in a direction perpendicular to the longitudinal direction of the monopolar 64 and the discharge portion 65. In the first embodiment, four gripped portions 76 are mounted on one monopolar 64 and one discharge portion 65. The four gripped portions 76 are arranged in a cross shape around the monopolar 64 and the discharge portion 65.

The wiring fixing portion 7 fixes wiring 66 extending from the monopolar 64. The wiring fixing portion 7 is provided at a place away from the surgical tool placement portion 6. The wiring fixing portion 7 is integrated with the surgical tool placement portion 6 by a connection portion 10 extending from the first outer edge 61 of the surgical tool placement portion 6. Voltage is applied to the monopolar 64 via the wiring 66.

As illustrated in FIG. 5, the wiring 66 has deflection in a state where the monopolar 64 is inserted into the first insertion portion 63a. In other words, the length of the wiring 66 between the monopolar 64 and the wiring fixing portion 7 is longer than a distance L1 between the first insertion portion 63a and the wiring fixing portion 7.

The piping fixing portion 8 fixes piping 67 extending from the discharge portion 65. The piping fixing portion 8 is provided at a place away from the surgical tool placement portion 6. The piping fixing portion 8 is integrated with the surgical tool placement portion 6 by the connection portion 10 extending from the first outer edge 61 of the surgical tool placement portion 6. The physiological saline is supplied to the discharge portion 65 through the piping 67. The piping 67 is formed of a flexible member.

As illustrated in FIG. 6, the piping 67 has deflection in a state where the discharge portion 65 is inserted into the second insertion portion 63b. In other words, the length of the piping 67 between the discharge portion 65 and the piping fixing portion 8 is longer than a distance L2 between the second insertion portion 63b and the piping fixing portion 8.

A clip 68, which is a surgical tool, is placed on the surgical tool placement portion 6. The clip 68 is a surgical tool that performs hemostasis or the like by clipping a blood vessel. The clip 68 is used while being gripped by the end effector 31. A pen 69, which is a surgical tool, is placed on the surgical tool placement portion 6. The pen 69 is a surgical tool that performs marking or the like on a portion to be incised. The pen 69 is used while being gripped by the end effector 31. A background sheet 70, which is a surgical tool, is placed on the surgical tool placement portion 6. The background sheet 70 is a surgical tool that is placed on the surgical site to facilitate viewing of the background. The background sheet 70 is used while being gripped by the end effector 31. A needle 71 is placed on the surgical tool placement portion 6. The needle 71 is a surgical tool used for suturing a blood vessel or the like. The needle 71 is used while being gripped by the end effector 31.

Here, the region of the surgical tool placement portion 6 is divided into a central region 6a that is a central portion in the circumferential direction of the first outer edge 61, a first end region 6b adjacent to the central region 6a, and a second end region 6c that is on the opposite side of the first end region 6b with the central region 6a interposed therebetween.

The first insertion portion 63a and the second insertion portion 63b are formed in the central region 6a. That is, the monopolar 64 and the discharge portion 65 are placed on the central region 6a. In addition, the clip 68, the pen 69, and the background sheet 70 are placed on the first end region 6b. Furthermore, in the first end region 6b, the background sheet 70 is placed at a place closer to the first outer edge 61 than the clip 68 and the pen 69. The needle 71 is disposed in the second end region 6c.

The extension portion 9 is formed to extend outward from the first outer edge 61. The extension portion 9 is formed to extend to both sides with the surgical tool placement portion 6 interposed therebetween. By fixing the extension portion 9 to the drape 22 with a tape 23, the plate 2 is fixed near the surgical site.

Note that, a gauze 72 is placed on the opposite side of the surgical tool placement portion 6 with the surgical site interposed therebetween.

### 1-1-3. Configuration of Operating Device

Returning to FIG. 1, the operating device 5 includes an image display unit 51 and an operation unit 52. An image captured by the camera 16 of the operation device 1 is displayed on the image display unit 51. The operation unit 52 is operated, so that the tool unit 15 of the operation device 1 can be operated. Accordingly, the posture of the tool driving unit 27 can be changed. In addition, the operation unit 52 is operated, so that the tool driving unit 27 can be operated to operate the end effector 31. The practitioner operates the operation unit 52 while checking the image displayed on the image display unit 51, and performs surgery on a patient at a distant place by the operation device 1. In addition, the operation unit 52 is operated, so that physiological saline can be discharged from the discharge portion 65. In addition, the operation unit 52 is operated, so that the camera 16 and the tool unit 15 can be rotated about the drive shaft 24 at the distal end of the arm 13.

### 1-2. Effects of First Embodiment

The surgical tool placement portion 6 of the plate 2 is surrounded by the first outer edge 61 and the second outer edge 62. That is, since the surgical tool placement portion 6 is formed in an annular shape, the surgical tool used while being gripped by the end effector 31 can be disposed so as to surround the surgical site. Therefore, the distance between the surgical site and the surgical tool can be reduced, and the operation distance of the tool driving unit 27 when gripping the surgical tool can also be reduced. As a result, the operation time can be shortened.

In addition, since the end effector 31 does not have a function of incising the surgical site like the monopolar 64 and is placed on the surgical tool placement portion 6 as a surgical tool, it is not necessary to replace the tool 30 at the time of incision. More specifically, during use of the tool 30 having the end effector 31 capable of gripping the surgical tool, if the monopolar 64 is gripped by the end effector 31, the incision can be performed without replacing the tool 30. That is, it is possible to shorten the operation time by reducing the time required for replacing the tool 30. In addition, since some of the surgical tools are placed on the surgical tool placement portion 6 without providing the tool 30 with the functions of the plurality of surgical tools, the tool 30 provided at the distal end of the arm 13 can be downsized. Note that, the tool 30 attached to the tool driving unit 27 may be provided with the end effector 31 having a function of a surgical tool different from the surgical tool placed on the surgical tool placement portion 6.

In addition, since the plurality of insertion portions 63 is provided, if the number of surgical tools to be inserted into the insertion portion 63 is smaller than the number of the insertion portions 63, the arrangement positions of the monopolar 64, the discharge portion 65, and the like can be selected according to the preference of the practitioner. In addition, when the monopolar 64, the discharge portion 65, or the like that has been used is returned to the insertion portion 63, the insertion portion 63 to which the monopolar 64, the discharge portion 65, or the like can be easily returned can be selected and returned according to the situation. As a result, it is possible to provide an environment in which the practitioner can easily perform the operation.

In addition, since the insertion portion 63 is a hole having a bottom, there is no risk that the surgical tool inserted into the insertion portion 63 penetrates the insertion portion 63 and damages the patient.

In addition, the surgical tool is inserted into the insertion portion 63, so that the surgical tool can be placed on the surgical tool placement portion 6 in a vertical arrangement. Since the vertical arrangement has a smaller planar area than the horizontal arrangement, it is possible to place many surgical tools compactly. Note that, even in a case where the surgical tools are placed in a horizontal arrangement, it is possible to obtain an effect that the surgical tools can be placed so as to surround the periphery of the surgical site as long as the surgical tool placement portion 6 has an annular shape.

In addition, since the monopolar 64 is provided in an inclined manner, the visibility of the monopolar 64 through the camera 16 is improved in the vertical arrangement. Therefore, the monopolar 64 can be easily gripped by the end effector 31. Even in a case where the monopolar 64 is not inclined, it is possible to obtain an effect that the surgical tools can be compactly placed as long as the monopolar is placed in a vertical arrangement.

In addition, since the gripped portion 76 is formed in the monopolar 64 and the discharge portion 65, the monopolar 64 and the discharge portion 65 are easily gripped by the end effector 31. In addition, since the monopolar 64 and the discharge portion 65 can be gripped more stably by gripping the end effector than by gripping the portion extending in the longitudinal direction of the monopolar 64 and the discharge portion 65, the gripping position is less likely to be displaced or the monopolar 64 and the discharge portion 65 is less likely to be dropped, and the surgery can be performed more safely.

In addition, since the wiring 66 extending from the monopolar 64 is bent between the wiring fixing portion 7 and the monopolar 64 while being fixed to the wiring fixing portion 7, a load from the wiring 66 is less likely to be applied to the monopolar 64. For example, even when some loads from the wiring 66 are applied to the side opposite to the monopolar 64 with the wiring fixing portion 7 interposed therebetween, the load is less likely to be applied to the monopolar 64, and it is possible to prevent the operation or the like of the monopolar 64 from being adversely affected.

In addition, since the piping 67 extending from the discharge portion 65 is bent between the piping fixing portion 8 and the discharge portion 65 while being fixed to the piping fixing portion 8, a load from the piping 67 is less likely to be applied to the discharge portion 65. For example, even when some loads from the piping 67 are applied to the side opposite to the discharge portion 65 with the piping fixing portion 8 interposed therebetween, the load is less likely to be applied to the discharge portion 65, and it is possible to prevent the operation or the like of the discharge portion 65 from being adversely affected.

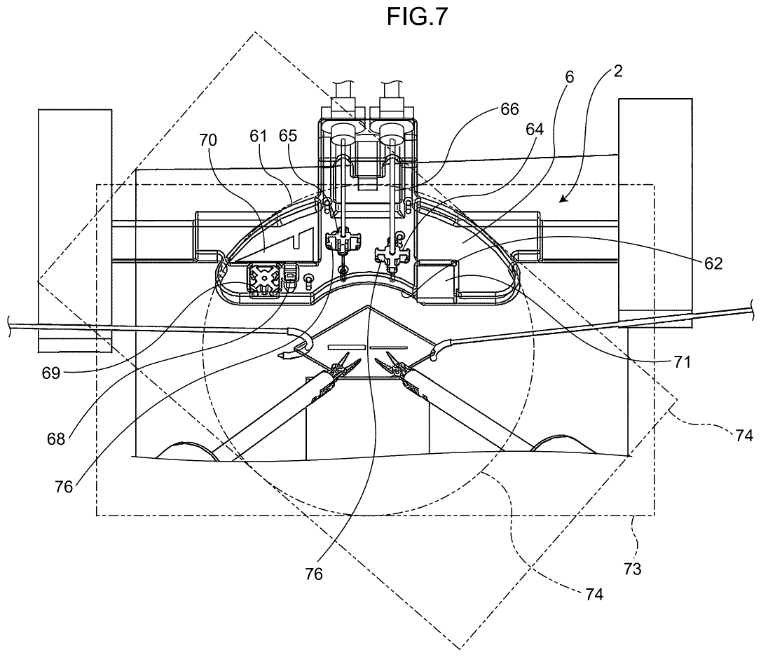

FIG. 7 is a partially enlarged plan view in which an affected part on which the plate according to the first embodiment is disposed is enlarged, and is a view for explaining a change in an imaging range due to rotation of a camera. In the surgery using the surgery supporting robot 100, there is a case where the camera 16 and the tool unit 15 are rotated about the drive shaft 24 according to the situation. In FIG. 7, an imaging range 73 captured by the camera 16 before rotation and displayed on the image display unit 51 and an imaging range 74 captured by the camera 16 after rotation and displayed on the image display unit 51 are indicated by two-dot chain lines. In a case where the imaging range 73 is rectangular, the region displayed in common regardless of the rotation angle of the camera 16 is a circular common region 75. In FIG. 7, the common region 75 is also indicated by a two-dot chain line.

As in the first embodiment, the annular surgical tool placement portion 6 between the arc-shaped first outer edge 61 and second outer edge 62 can fall within the circular common region 75 as illustrated in FIG. 7. Therefore, even if the camera 16 is rotated, the surgical tool placed on the surgical tool placement portion 6 can be displayed on the image display unit 51 at all times. Therefore, it is not necessary to change the position of the plate 2 so that the surgical tool falls within the imaging range each time the camera 16 is rotated, and the operation time can be shortened.

### 2. Supplementary Note

Note that, the present technology can also have the following configurations.
(1). A plate comprising
   a surgical tool placement portion that is a region on which a surgical tool is placed and sandwiched between an arc-shaped first outer edge and an arc-shaped second outer edge concentric with the first outer edge and having a small radius.
(2). The plate according to (1),
   wherein a plurality of insertion portions extending in a thickness direction of the surgical tool placement portion and into which a distal end of the surgical tool is inserted is formed.
(3). The plate according to (2),
   wherein the insertion portion includes a first insertion portion into which a distal end of an electric scalpel that is the surgical tool is inserted.
(4). The plate according to (3), further comprising:
   the electric scalpel that is the surgical tool having a distal end inserted into the first insertion portion; and
   a wiring fixing portion that fixes wiring extending from the electric scalpel,
   wherein a length of the wiring between the wiring fixing portion and the electric scalpel is longer than a distance between the wiring fixing portion and the first insertion portion.
(5). The plate according to (4),
   wherein the electric scalpel is disposed to be inclined so as to be away from a center of the first outer edge as the electric scalpel is away from a surface of the surgical tool placement portion.
(6). The plate according to any one of (2) to (5),
   wherein the insertion portion includes a second insertion portion into which a distal end of a discharge portion that is the surgical tool and discharges physiological saline is inserted.
(7). The plate according to (6), further comprising:
   the discharge portion having a distal end inserted into the second insertion portion; and
   a piping fixing portion that fixes a piping extending from the discharge portion,
   wherein a length of the piping between the piping fixing portion and the discharge portion is longer than a distance between the piping fixing portion and the second insertion portion.
(8). The plate according to any one of (1) to (7), further comprising
   an extension portion extending from the first outer edge toward an outside of the surgical tool placement portion.
(9). The plate according to any one of (1) to (8), further comprising
   a clip that is the surgical tool.
(10). The plate according to any one of (1) to (9), further comprising
   a pen that is the surgical tool.
(11). The plate according to any one of (1) to (10), further comprising
   a needle that is the surgical tool.
(12). The plate according to (1),
   wherein a plurality of insertion portions extending in a thickness direction of the surgical tool placement portion and into which a distal end of the surgical tool is inserted is formed,
   the insertion portion includes a first insertion portion into which a distal end of an electric scalpel that is the surgical tool is inserted, and a second insertion portion into which a distal end of a discharge portion that is the surgical tool and discharges physiological saline is inserted,
   the plate further comprising a clip, a pen, and a needle that are the surgical tools,
   wherein the first insertion portion and the second insertion portion are formed in a central region that is a central portion in a circumferential direction of the first outer edge,
   the clip and the pen are disposed in a first end region adjacent to the central region in the circumferential direction, and
   the needle is disposed in a second end region opposite to the first end region with the central region interposed therebetween in the circumferential direction.
(13). The plate according to (12), further comprising
   a background sheet that is the surgical tool,
   wherein the background sheet is disposed in the first end region and closer to the first outer edge than the clip and the pen.
(14). The plate according to (2), further comprising
   a gripped portion protruding in a direction perpendicular to a longitudinal direction of the surgical tool.
(15). A surgical robot system comprising:
   a plate according to any one of (1) to (14); and
   a surgery supporting robot including an end effector that grips the surgical tool and a camera that captures an image of a surgical site on which the plate is installed.
(16). The surgical robot system according to (15),
   wherein the surgery supporting robot includes a drive shaft that rotates an imaging range of the camera, and
   the surgical tool placement portion falls within the imaging range even when the camera is rotated.

### Reference Signs List

1 OPERATION DEVICE
2 PLATE
5 OPERATING DEVICE
6 SURGICAL TOOL PLACEMENT UNIT
6a CENTRAL REGION
6b FIRST END REGION
6c SECOND END REGION
7 WIRING FIXING PORTION
8 PIPING FIXING PORTION
9 EXTENSION PORTION
10 CONNECTION PORTION
11 BASE
12 SUPPORT COLUMN
13 ARM
15 TOOL UNIT
16 CAMERA
17 TOOL PROVIDING UNIT
21 ROTATION SHAFT
22 DRAPE
22a OPENING
23 TAPE
24 DRIVE SHAFT
25 BASE PORTION
27 TOOL DRIVING UNIT
28 FIRST DRIVE SHAFT
29 SECOND DRIVE SHAFT
30 TOOL
31 END EFFECTOR
51 IMAGE DISPLAY UNIT
52 OPERATION UNIT
60 CENTER
61 FIRST OUTER EDGE
62 SECOND OUTER EDGE
63 INSERTION PORTION
63a FIRST INSERTION PORTION
63b SECOND INSERTION PORTION
64 MONOPOLAR
65 DISCHARGE PORTION
66 WIRING
67 PIPING
68 CLIP
69 PEN
70 BACKGROUND SHEET
71 NEEDLE
72 GAUZE
73, 74 IMAGING RANGE
75 COMMON REGION
76 GRIPPED PORTION
100 SURGERY SUPPORTING ROBOT

1. A plate comprising
a surgical tool placement portion that is a region on which a surgical tool is placed and sandwiched between an arc-shaped first outer edge and an arc-shaped second outer edge concentric with the first outer edge and having a small radius.

2. The plate according to claim 1,
wherein a plurality of insertion portions extending in a thickness direction of the surgical tool placement portion and into which a distal end of the surgical tool is inserted is formed.

3. The plate according to claim 2,
wherein the insertion portion includes a first insertion portion into which a distal end of an electric scalpel that is the surgical tool is inserted.

4. The plate according to claim 3, further comprising:
the electric scalpel that is the surgical tool having a distal end inserted into the first insertion portion; and
a wiring fixing portion that fixes wiring extending from the electric scalpel,
wherein a length of the wiring between the wiring fixing portion and the electric scalpel is longer than a distance between the wiring fixing portion and the first insertion portion.

5. The plate according to claim 4,
wherein the electric scalpel is disposed to be inclined so as to be away from a center of the first outer edge as the electric scalpel is away from a surface of the surgical tool placement portion.

6. The plate according to claim 2,
wherein the insertion portion includes a second insertion portion into which a distal end of a discharge portion that is the surgical tool and discharges physiological saline is inserted.

7. The plate according to claim 6, further comprising:
the discharge portion having a distal end inserted into the second insertion portion; and
a piping fixing portion that fixes a piping extending from the discharge portion,
wherein a length of the piping between the piping fixing portion and the discharge portion is longer than a distance between the piping fixing portion and the second insertion portion.

8. The plate according to claim 1, further comprising
an extension portion extending from the first outer edge toward an outside of the surgical tool placement portion.

9. The plate according to claim 1, further comprising
a clip that is the surgical tool.

10. The plate according to claim 1, further comprising
a pen that is the surgical tool.

11. The plate according to claim 1, further comprising
a needle that is the surgical tool.

12. The plate according to claim 1,
wherein a plurality of insertion portions extending in a thickness direction of the surgical tool placement portion and into which a distal end of the surgical tool is inserted is formed,
the insertion portion includes a first insertion portion into which a distal end of an electric scalpel that is the surgical tool is inserted, and a second insertion portion into which a distal end of a discharge portion that is the surgical tool and discharges physiological saline is inserted,
the plate further comprising a clip, a pen, and a needle that are the surgical tools,
wherein the first insertion portion and the second insertion portion are formed in a central region that is a central portion in a circumferential direction of the first outer edge,
the clip and the pen are disposed in a first end region adjacent to the central region in the circumferential direction, and
the needle is disposed in a second end region opposite to the first end region with the central region interposed therebetween in the circumferential direction.

13. The plate according to claim 12, further comprising
a background sheet that is the surgical tool,
wherein the background sheet is disposed in the first end region and closer to the first outer edge than the clip and the pen.

14. The plate according to claim 2, further comprising
a gripped portion protruding in a direction perpendicular to a longitudinal direction of the surgical tool.

15. A surgical robot system comprising:
a plate according to claim 1; and
a surgery supporting robot including an end effector that grips the surgical tool and a camera that captures an image of a surgical site on which the plate is installed.

16. The surgical robot system according to claim 15,
wherein the surgery supporting robot includes a drive shaft that rotates an imaging range of the camera, and
the surgical tool placement portion falls within the imaging range even when the camera is rotated.
